(19) United States
(12) Patent Application Publication
(10) Pub. No.: US 2019/0201002 A1
(43) Pub. Date: Jul. 4, 2019

(54) BALLOON CATHETER DEVICE FOR USE IN AN AORTA OF A RABBIT OR A HUMAN BEING

(71) Applicant: Medizinische Universität Graz, Graz (AT)

(72) Inventors: Peter OPRIESSNIG, Graz (AT); Gerd HÖRL, Graz (AT); Wolfgang SAX, Graz (AT); Gunter ALMER, Graz (AT); Harald MANGGE, Graz (AT)

(21) Appl. No.: 16/327,377

(22) PCT Filed: Aug. 24, 2017
(86) PCT No.: PCT/EP2017/071300

§ 371 Date: Feb. 22, 2019

(30) Foreign Application Priority Data
Aug. 25, 2016 (GB) 1614500.5

(51) Int. Cl. A61B 17/12; A61M 25/10; A61M 29/02; A61D 1/00; A61B 17/22

(52) U.S. Cl. CPC A61B 17/12109; A61B 17/1204; A61M 25/10182; A61M 25/0113; A61M 29/02; A61D 1/00; A61B 17/22032; A61M 25/10185

(57) ABSTRACT

The invention relates to a balloon catheter device (1) for use in an aorta of a rabbit or a human being. The device (1) comprises a balloon catheter (2), a syringe (3), an actuator (4), a pressure sensor (5), a balloon retracting unit (6), a first control unit (7) and a second control unit (8). The balloon catheter (2) comprises a flexible tube (10) and a balloon (11) at a distal end (12) of the tube (10) and the syringe (3) comprises a chamber (16) containing a fluid, wherein the chamber (16) is connected to a proximal end of the tube (10). The actuator (4) is adapted for increasing/decreasing a volume of the fluid contained within the balloon (11). The pressure sensor (5) is adapted for sensing an inflation pressure of the balloon (11) and the balloon retracting unit (6) is adapted for retracting the balloon (11) out of the aorta. The first control unit (7) is adapted to receive pressure data representing the inflation pressure of the balloon (11) measured by the pressure sensor (5) and to control the actuator (4) depending on the received pressure data, such that the inflation pressure of the balloon (11) is kept stable at a predefined inflation pressure value, and the second control unit (8) is adapted to control the balloon retracting unit (6), such that the balloon (11) is retracted out of the aorta at a constant speed.

### FIELD OF THE INVENTION

The invention relates to a balloon catheter device for use in an aorta of a rabbit or a human being. In particular, the present invention deals with the development of a standardized balloon injury device for the generation of reproducible minimal invasive-lesions in rabbits (pressure controlled balloon injury). At the same time the device should also be adapted for being used safely in balloon thrombectomy.

### BACKGROUND OF THE INVENTION

In the field of pre-clinical atherosclerosis (AS) research, New Zealand white (NZW) rabbits are the standard model to investigate plaque development and therapy. Importantly, atherosclerotic vascular lesions of these animals are closer to human pathology then example given lesions of ApoE deficient mice. Typically, abdominal aortic AS is induced by a combination of a balloon catheter denudation and a high-fat diet. As the abdominal aorta is markedly narrowing from proximal to distal (5 to 3 mm in diameter) a constant balloon volume (manual balloon injury) introduces significant pressure differences (1 to 3 bar) when retracted. Consequently, a heterogeneous plaque development is observed.

By now, manual balloon injury is performed from the iliac bifurcation to the renal artery (or take arch) or vice versa introducing a balloon catheter through the carotid artery or femoral artery, respectively. Respective methods are described e.g. in the article “detection of new vessels in atherosclerotic plaques of rabbits using dynamic contrast enhanced MRI and 18 F—FDG PET” by Calcagno et al, published in the journal “arteriosclerosis, thrombosis, and vascular biology—Journal of the American Heart Association” on 8 May 2008, or in the article “Reproducibility of black blood dynamic contrast enhanced magnetic resonance imaging in aortic plaques of atherosclerotic rabbits” by Calcagno et al, published in the “Journal of magnetic resonance imaging 32; 191-198 (2010)” in 2010.

It is known to inflate the balloon manually using a liquid or gas filled syringe. During the passage of the catheter, inflation pressure variations are introduced. Effects of inflation pressure on plaque development are the topic of the article “effects of inflation pressure of balloon catheter on vascular injuries and subsequent development of intimai hyperplasia in rabbit aorta” by Asada et al, published in Atherosclerosis 121 (1996) 45-53 in 1996.

Regarding balloon thromboembolectomy in acute arterial occlusion, there is a need for an improved technique which especially helps junior doctors to manage such a surgery in less training time and, more important, improves patient safety and potentially lower catheter induced material injuries. This is very important as the procedure is the most frequently required emergency procedure in vascular surgery, for example with more than 20,000 operations per year in Germany.

### SUMMARY OF THE INVENTION

It is an object of the present invention to provide a balloon catheter device for use in an aorta of a rabbit or a human being which enables inducing homogeneous and reproducible injuries in the aorta of the rabbit and to avoid injuries in the aorta of the human being.

The problem is solved by the subject matter according to the independent claims. The dependent claims, the following description and the drawings show preferred embodiments of the invention.

One core of the invention lies in that the balloon catheter device according to the present invention comprises means which are adapted for automatically adapting a volume within a balloon of a balloon catheter to changing diameters of an aorta of a rabbit or a human being into which the balloon has been inserted. By this, an inflation pressure of the balloon and a pressure respectively force which the balloon exerts on the aorta of the rabbit of the human being can be controlled, in particular in such a way that said pressure respectively force is kept stable at a predefined value. Furthermore, the balloon catheter device comprises means which are adapted for pulling out or retracting the balloon out of the aorta of the rabbit or the human being at a constant speed. The balloon catheter device further comprises a control circuit for controlling aforementioned means for adapting the balloon volume and for retracting the balloon out the aorta.

The combination of both effects, namely the stable pressure of the balloon and the constant retracting speed of the balloon out of the aorta, enables inducing homogeneous, standardized and reproducible injuries in the aorta of the rabbit respectively to avoid injuries in the aorta of the human being.

According to an aspect of the invention, a balloon catheter device for use in an aorta of a rabbit or a human being is provided. The balloon catheter device comprises a balloon catheter, a syringe, an actuator, a pressure sensor, a balloon retracting unit, a first control unit and a second control unit.

The balloon catheter comprises a flexible tube and a balloon at a distal end of the tube, and the syringe comprises a chamber containing a fluid, wherein the chamber is connected to a proximal end of the tube. The fluid within the chamber of the syringe can be liquid, e.g. NaCl, or gaseous. The whole system, i.e. the chamber of the syringe, the flexible tube and the balloon can be filled with the fluid.

The syringe can further comprise a housing and a rod with a plunger, wherein the plunger is disposed at a distal end of the rod. The chamber can be variable in its volume and can be defined by an inner wall of the housing of the syringe and the plunger. In a known manner, the rod can be slid into the housing of the syringe and pulled out of the housing of the syringe, thereby increasing the volume of the chamber (when being pulled out of the housing) or decreasing the volume of the chamber (when being slid into the housing).

The actuator is adapted for injecting the fluid contained in the chamber of the syringe into the flexible tube, thereby increasing a volume of the fluid contained within the balloon, and for sucking in fluid contained in the flexible tube into the chamber of the syringe, thereby decreasing the volume of the fluid contained within the balloon.

In particular, the actuator can be a linear actuator which can be adapted for linearly moving the rod of the syringe in a distal direction of the syringe for injecting the fluid contained in the chamber of the syringe into the flexible tube, thereby increasing a volume of the fluid contained within the balloon.

The linear actuator can further be adapted for linearly moving the rod of the syringe in a proximal direction of the syringe for sucking in fluid contained in the flexible tube into the chamber of the syringe, thereby decreasing the volume of the fluid contained within the balloon.

The pressure sensor is adapted for sensing an inflation pressure of the balloon. The pressure sensor can e.g. be situated or disposed within the flexible tube, where the pressure sensor can sense the pressure within the flexible tube and determine the pressure within the balloon. Alternatively, the pressure sensor can be disposed between the syringe and the flexible tube and can be in a fluid connection with both the syringe (i.e. its chamber) and the flexible tube.

The balloon retracting unit is adapted for retracting the balloon out of the aorta. For example, the balloon retracting unit can comprise means by which the flexible tube can be pulled in a direction such that the balloon at the end of the flexible tube is pulled out of the aorta.

The first control unit is communicatively connected to the pressure sensor and the actuator, in particular the linear actuator. Such a connection can be made by wires or wireless in a known manner. In particular, the first control unit can receive pressure data generated by the pressure sensor, wherein the pressure data represent measured balloon inflation pressures. Also, the first control unit can send command signals to the actuator, in particular the linear actuator.

The second control unit is communicatively connected to the balloon retracting unit. Such a connection can be made by wires or wireless in a known manner. In particular, the second control unit can send command signals to the balloon retracting unit.

The first control unit is adapted to receive pressure data representing the inflation pressure of the balloon measured by the pressure sensor and to control the actuator, in particular the linear actuator, depending on the received pressure data, such that the inflation pressure of the balloon is kept stable at a predefined inflation pressure value. In this context, the term “stable” can in an ideal case mean that the inflation pressure of the balloon is kept constant at a predefined inflation pressure value. In reality, the inflation pressure of the balloon may deviate around the predefined balloon inflation pressure in a certain range.

In particular, the first control unit can receive pressure data from the pressure sensor according to which the pressure within the balloon is rising. This can be due to a decrease in the diameter of the aorta of the rabbit or the human being into which the balloon has been inserted. The first control unit can control the actuator, such that the volume of the chamber of the syringe is increased. In particular, the first control unit can control the linear actuator, such that the linear actuator pulls the rod out of the housing of the syringe, thereby increasing the volume of the chamber. As a result, a vacuum is built within the chamber which forces fluid contained within the flexible tube to move into the chamber. However, the volume of fluid contained within the flexible tube remains constant. As the total volume of fluid contained within the chamber, the flexible tube and the balloon is always constant, fluid contained within the balloon, which is flexible, is forced to move out of the balloon and into the flexible tube. Thereby, the volume of the flexible balloon is decreased. By this, the volume of the balloon is being adapted to a decreased diameter of the aorta in order to stabilize the inflation pressure of the balloon respectively the pressure within the balloon, wherein a pressure respectively force which the balloon exerts on the aorta is also being adapted in a way that it is kept stable at an intended value.

In an opposite case, the first control unit can receive pressure data from the pressure sensor according to which the pressure within the balloon is falling. This can be due to an increase in the diameter of the aorta of the rabbit of the human being into which the balloon has been inserted. The first control unit can control the actuator, such that the volume of the chamber of the syringe is decreased. In particular, the first control unit can control the linear actuator, such that the linear actuator pushes the rod deeper into the housing of the syringe, thereby decreasing the volume of the chamber. As a result, fluid contained within the chamber is forced to leave the chamber and to flow into the flexible tube. As a consequence, less fluid is contained within the chamber, wherein the volume of fluid contained within the flexible tube is constant. As the total volume of fluid contained within the chamber, the flexible tube and the balloon is constant, the volume of the flexible balloon is increased. By this, the volume of the balloon is being adapted to an increased diameter of the aorta in order to stabilize the inflation pressure of the balloon respectively the pressure within the balloon, wherein a pressure respectively force which the balloon exerts on the aorta is also being adapted in a way that it is kept stable at an intended value.

Furthermore, the second control unit is adapted to control the balloon retracting unit, such that the balloon is retracted out of the aorta at a constant speed. As an overall result, the balloon can be pulled out of the aorta of the rabbit of the human being at a constant speed and at a stable predetermined inflation pressure. By this, the aorta of the rabbit can be damaged in such a way that homogenous plaques can be induced on the one hand, and on the other hand, the aorta of the human being can be saved from damages when pulling out the balloon.

According to an embodiment, the balloon catheter is a balloon catheter for generating reproducible minimal-invasive lesions in the aorta of the rabbit, wherein the predefined inflation pressure value is high enough to cause the balloon to generate intended lesions in the aorta of the rabbit, if the balloon has an inflation pressure as high as the predefined inflation pressure value.

According to another embodiment, the balloon catheter is a balloon catheter for use in pressure controlled balloon thrombectomy on the human being, wherein the predefined inflation pressure value is not high enough to cause the balloon to generate lesions in the aorta of the human being, if the balloon has an inflation pressure as high as the predefined inflation pressure value.

The pressure sensor can be a Luer lock pressure sensor. The Luer lock pressure sensor can be connected to a Luer lock fitting of the syringe on the one side and to the flexible tube on the other side. The Luer lock pressure sensor is comfortable to and can be combined with lots of different types of syringes because the Luer lock fitting is standardized.

The balloon catheter device can further comprise an encoder and recording unit and a quadrature encoder, wherein the encoder and recording unit is adapted for receiving, encoding and storing pressure data representing measured pressures from the pressure sensor and step data representing a number of steps performed by the actuator, in particular by the linear actuator, from the quadrature encoder.

The encoder and recording unit can be adapted for calculating a volume of the balloon depending on the received pressure data and step data.

The balloon catheter device can further comprise a computer unit, wherein the encoder and recording unit is adapted for transmitting the received pressure data and step data to the computer unit, and wherein the computer unit is adapted for storing the transmitted pressure data and step data and for creating real-time plots of both the transmitted pressure data and step data.

According to another embodiment, the first control unit and the second control unit are integrated into a single control unit. Furthermore, the encoder and recording unit can also be integrated into the single control unit.

According to another embodiment, the balloon retracting unit comprises a rack, a sled, and driving means, wherein the sled is slideably mounted on the rack, and wherein the syringe and the linear actuator are fixedly mounted on the sled. The driving means are adapted for driving the sled such that the sled is sliding along the rack at a sliding speed, and the second control unit can control the driving means, such that the sliding speed of the sled is constant.

The rack can comprise two guiding rods, wherein the driving means can comprise a rotating stepper and a spindle drive. The sled can be guided in a linear direction by means of the two guiding rods, wherein the spindle drive can drive the sled such that the sled is sliding along the two guiding rods at the sliding speed, and wherein the second control unit can control the spindle drive, such that the sliding speed of the sled is constant.

According to another embodiment, the balloon retracting unit comprises a retracting roll assembly, and an electric motor, in particular a DC gear motor. The electric motor is adapted for driving the retraction roll assembly such that the retracting roll assembly is rotating at a rotating speed, wherein the retracting roll assembly is configured for advancing the flexible tube of the balloon catheter such that the flexible tube is retracted out of the aorta when driven by the electric motor, and wherein the second control unit is configured for controlling the electric motor, such that the rotating speed of the retracting roll assembly is constant.

According to another embodiment, the first control unit comprises a memory unit, wherein a predefined upper limit value of the balloon inflation pressure is stored in the memory unit, wherein a predefined lower limit value of the balloon inflation pressure is stored in the memory unit, and wherein a predefined actuating speed of the linear actuator is stored in the memory unit.

The first control unit is adapted for deflating the balloon by driving the linear actuator at the predefined actuating speed, such that the rod of the syringe is moved in a proximal direction of the syringe at a speed proportional to the predefined actuating speed and sucks in fluid contained in the flexible tube into the chamber of the syringe, thereby decreasing the volume of the fluid contained within the balloon, if a value of a measured inflation pressure is higher than the predefined lower limit value of the balloon inflation pressure.

The first control unit is also adapted for inflating the balloon by driving the linear actuator at the predefined actuating speed, such that the rod of the syringe is moved in a distal direction of the syringe at a speed proportional to the predefined actuating speed and injects the fluid contained in the chamber of the syringe into the flexible tube, thereby increasing a volume of the fluid contained within the balloon, if the value of the measured inflation pressure is lower than the predefined upper limit value of the balloon inflation pressure.

Furthermore, the first control unit is adapted for inflating the balloon, if the value of the measured inflation pressure is higher than the predefined lower limit value of the balloon inflation pressure and if the value of the measured inflation pressure is lower than the predefined upper limit value of the balloon inflation pressure.

In another embodiment, the pressure sensor is adapted to sense the inflation pressure of the balloon in real-time. The balloon catheter device can be adapted to transmit the inflation pressure of the balloon wirelessly to an external computer unit, e.g. by means of a suitable interface. Preferably, the balloon catheter device is adapted to transmit the inflation pressure of the balloon immediately after the inflation pressure has been sensed. This embodiment allows a monitoring of the inflation pressure of the balloon in real-time. This helps a vascular doctor to avoid injuries of the vascular wall caused by a too high contact pressure of the balloon during surgery.

Furthermore, data representing the inflation pressure or multiple inflation pressures of the balloon can be recorded in the external computer unit, e.g. on a suitable storing unit. The recorded data enables a user of the balloon catheter device, e.g. a vascular practitioner, to use these additional data and to evaluate if they correlate with results of follow-up inquiries and the frequency of complications. Additionally, said data, which has been collected in vascular surgery, contribute to determine an optimal inflation pressure of the balloon for this specific type of surgery, and therefore allows to lower the individual surgery risk for the patient and to avoid late damages up to a loss of extremities.

These and other aspects of the invention will be apparent from and elucidated with reference to the embodiments described hereinafter.

### BRIEF DESCRIPTION OF THE DRAWINGS

In the following description, exemplary embodiments of the invention are explained with reference to the accompanying schematic drawing in which

FIG. 1 shows a top view of a device in accordance with a first embodiment of the invention,

FIG. 2 shows a top view of a device in accordance with a second embodiment of the invention and

FIG. 3 shows a diagram illustrating a chronological sequence of a balloon inflation pressure being controlled by the device according to FIG. 1 or 2.

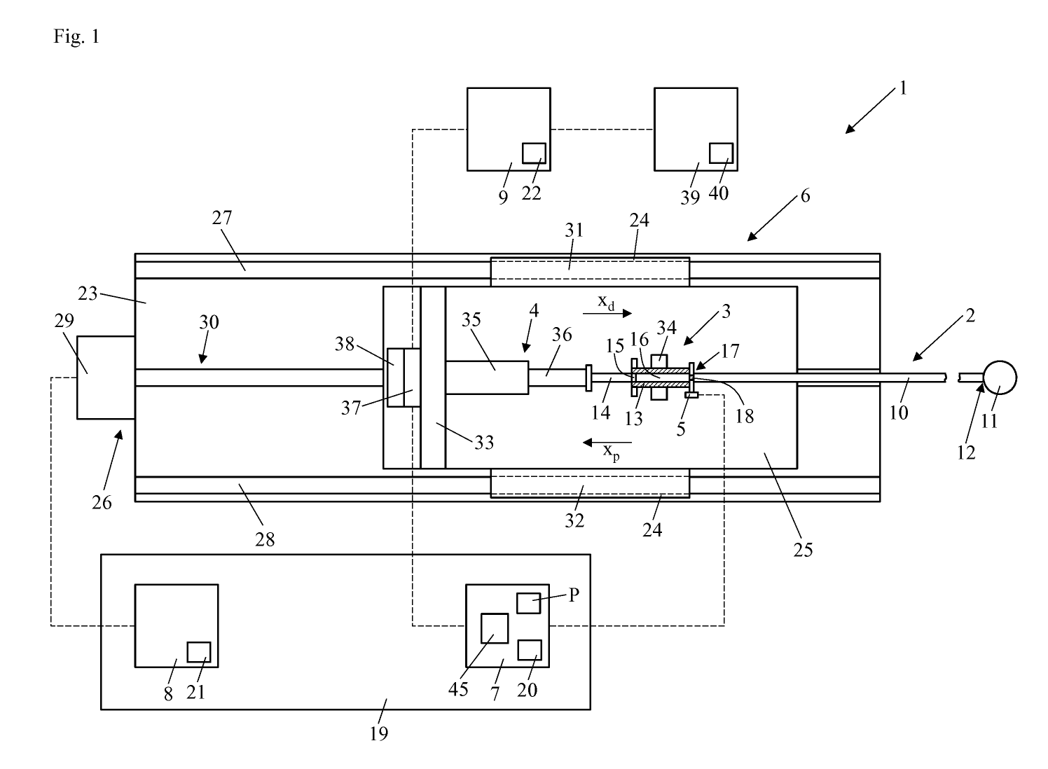

FIG. 1 shows a balloon catheter device 1 for use in an aorta of a rabbit (not shown) or a human being (not shown) in accordance with an embodiment of the present invention. As described in the following, the balloon catheter device 1 can be adapted for generating reproducible minimal invasive lesions in the aorta of the rabbit or for being used in pressure controlled balloon thrombectomy on the human being.

The balloon catheter device 1 comprises a balloon catheter 2, a syringe 3, a linear actuator 4, a pressure sensor 5, a balloon retracting unit 6, a first control unit 7, a second control unit 8 and an encoder and recording unit 9.

The balloon catheter 2 comprises a flexible tube 10 and a balloon 11 at a distal end 12 of the flexible tube 10. The balloon 11 is also flexible, meaning that it can be inflated by filling it with a fluid and deflated by sucking the fluid out of it. The flexible tube 10 is in a fluid connection with the balloon 11. In particular, a fluid contained in the flexible tube 10 can be pushed into the balloon 11 and vice versa in order to inflate or deflate the balloon 11. The flexible tube 10 and the balloon 11 are adapted to be inserted into the aorta of the rabbit and the human being. The balloon catheter 2 can be positioned within the aorta, wherein the balloon 11 is in a deflated state. When brought into position in the aorta of the rabbit, the balloon 11 can be inflated with sufficient pressure and be retracted by retracting the flexible tube 10, thereby causing a minimal-invasive lesion in the aorta of the rabbit, as will be described further below. Furthermore, when brought into position in the aorta of the human being, the balloon 11 can be inflated with sufficient but not too high pressure and be retracted by retracting the flexible tube 10, such that an occlusion respectively a thrombus within the aorta of the human being can be removed without causing an invasive lesion in the aorta of the human being, as will be described further below.

The syringe 3 comprises a housing 13 in which a rod 14 having a plunger 15 can slide in opposite directions, namely in a proximal direction xₚ and in a distal direction x_{d} of the syringe 3, thereby increasing or decreasing a volume of a chamber 16 within the housing 13. The chamber 16 is filled with a fluid and is connected to a Luer cone region 17 of the syringe 3. The flexible tube 10 and the balloon 11 are filled with the same fluid as the chamber 16, also. The fluid within the chamber 16, the flexible tube 10 and the balloon 11 can be liquid, e.g. NaCl, or gaseous. The Luer cone region 17 comprises an injection port 18 which is in a fluid communication with the chamber 16. A proximal end of the flexible tube 10 is fixedly connected to the Luer cone region 17 of the syringe 3, such that the chamber 16 is in a fluid connection with the flexible tube 10 via the injection port 18.

The pressure sensor 5 which is a Luer lock pressure sensor is mounted on the syringe 3 in the Luer cone region 17. The pressure sensor 5 is in a fluid connection with the flexible tube 10 on the site shown right in FIG. 1, and the injection port 18 on the site shown left in FIG. 1, such that the pressure sensor 5 senses a pressure within the flexible tube 10 which indicates the pressure in the balloon 11 of the balloon catheter 2. The pressure sensor 5 generates according pressure data which represents the inflation pressure of the balloon 11. The pressure sensor 5 continuously senses the pressure as described above and generates respective a number of pressure data.

The first control unit 7 and the second control unit 8 can be integrated into a single control unit 19 which fulfills all the functions of the first control unit 7 and the second control unit 8. Although not shown in FIG. 1, the encoder and recording unit 9 can also be integrated into the single control unit 19, wherein the single control unit 19 fulfils all the functions of the encoder and recording unit 9. The first control unit 7 can comprise a first memory unit 20, the second control unit 8 can comprise a second memory unit 21 and the encoder and recording unit 9 can comprise a third memory unit 22.

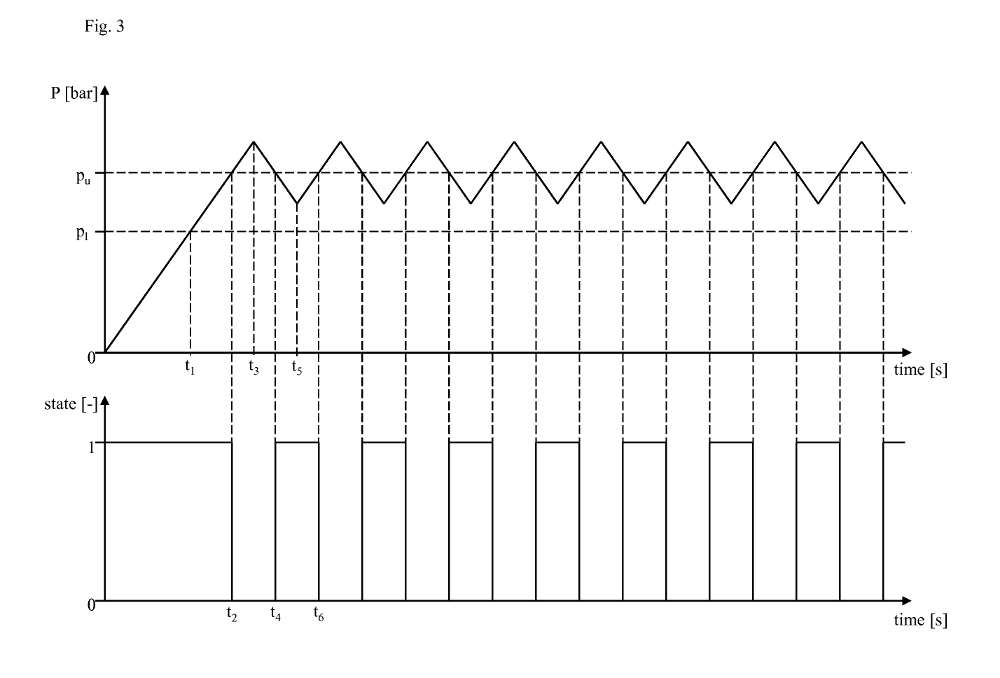

The first control unit 7 can example given be mounted on an Arduino UNO board. The first control unit 7 can comprise a stepper driver 45 (e.g. a DRV8825) to operate the linear actuator 4 respectively its driving unit 37 as described below. Furthermore, the first control unit 7 can comprise an external power supply (not shown) for the stepper driver and an “on/standby” button (not shown). If the “on/standby” button is pressed, the stepper driver 45 of the first control unit 7 controls the driving unit 37 in such a way that the balloon 11 is filled to a predefined balloon inflation pressure, wherein the predefined balloon inflation pressure is hold stable by a threshold technique as described in conjunction with FIG. 3 further below. By pressing a “go-home” button (not shown) of the first control unit 7 the stepper driver 45 of the first control unit 7 controls the driving unit 37 in such a way that stamp 36 of the linear actuator 4 is moved into a starting position, thereby emptying the balloon 11 up to a starting volume. Data representing said starting position can be predetermined and stored in the first memory unit 20. Additionally, the first control unit 7 can comprise an interface (not shown) for a connection with the pressure sensor 5 and an interface (not shown) for a connection with the linear actuator 4.

### Retracting of the Flexible Tube

The balloon retracting unit 6 according to the exemplary embodiment shown by FIG. 1 comprises a rack 23, a sled 24, a holder 25 and driving means 26. The rack 23 comprises two guiding rods 27 and 28. The driving means 26 comprise a rotating stepper 29 and a spindle drive 30.

The holder 25 is a part of and fixedly mounted on the sled 24 on an upper surface thereof. The sled 24 comprises parallel through-holes 31 and 32 (indicated by dashed lines) of such size and length that the two guiding rods 27, 28 can be pushed through the through-holes 31 and 32 and the sled 24 together with the holder 25 can linearly slide along the guiding rods 27, 28 in the proximal direction xₚ and in the distal direction x_{d}. By this, the sled 24 together with its holder 25 is slid ably mounted on the rack 23, wherein the sled 24 is guided in a linear direction, namely in the proximal direction xₚ and in the distal direction x_{d}, by means of the two guiding rods 27, 28.

The sled 24 together with its holder 25 can be driven, in particular can be slid along the two guiding rods 27, 29 of the rack 23 in the proximal direction xₚ and in the distal direction x_{d} as described above by the spindle drive 30 which, in turn, can be driven by the rotating stepper 29 of the driving means 26. In particular, the sled 24 can be driven by the spindle drive 30 at a constant speed. The second control unit 8 is communicatively connected to the rotating stepper 29 of the retracting unit 6. The second control unit 8 is adapted to control the balloon retracting unit 6, such that the balloon 11 of the balloon catheter to is retracted out of the aorta of the rabbit or the human being at a constant speed.

In particular, the second control unit 8 controls the rotating stepper 29 such that it is moved an intended number of steps in an intended turning direction, thereby causing the spindle drive 30 also to rotate an intended number of steps in an intended turning direction. The second control unit 8 controls the rotating stepper 29 such that it is moved the intended number of steps at a constant frequency, thus enabling a constant rotating speed of the spindle drive 30 as well as a constant sliding speed of the sled 24 together with its holder 25. As it will be described further below, the flexible tube 10 is fixed to the holder 25 of the sled 24. Therefore, if the sled 24 is driven by the spindle drive 30 in the proximal direction xₚ at a constant speed, the flexible tube 10 is also moved in the proximal direction xₚ at a constant speed. By this movement of the flexible tube 10 in the proximal direction xₚ, the flexible tube 10 and especially the balloon 11 of the balloon catheter 11 can be retracted out of the aorta of the rabbit or the human being by use of the balloon retracting unit 6, if the flexible tube 10 and the balloon 11 have been inserted into the aorta before.

The spindle drive 30 comprises an external thread (not shown) and is guided through a third through-hole (not shown) within the sled 24. The third through-hole runs parallel to the first through-hole 31 and to the second through-hole 32. The third through-hole comprises an internal thread (not shown) corresponding to the external thread of the spindle drive 30. The third through-hole is of such length and size that the spindle drive 30 can be pushed through the third through-hole. If the spindle drive 30 is rotating as described above, it drives the sled 24 together with its holder 25, such that the sled 24 slides along the rack 23 in the distal direction x_{d} (if the spindle drive is turning in a first rotating direction) or in the proximal direction xₚ (if the spindle drive is turning in a second direction, wherein the second rotating direction is opposite to the first rotating direction).

A respective algorithm to control the rotating stepper 29 is stored on the second memory unit 21 of the second control unit 8. For example, the sled can be driven at a constant speed of 5 mm/s. The inventor found out that such a speed is ideal because it enables a relatively fast retraction of the balloon 11 and the flexible tube 10 out of the aorta of the rabbit of the human being on the one hand, and on the other hand such a speed is not too high (which could negatively influence the first control unit 7 in that the first control unit 7 cannot adapt the volume respectively the inflation pressure of the balloon 11 fast enough, which could lead to an explosion of the balloon 11 or in the worst case of the aorta of the rabbit or the human being).

### Inflating (Filling)/Deflating (Emptying) of the Balloon

The linear actuator 4 is fixed to the holder 25 at a traverse 33 of the holder 25. The housing 13 of the syringe 3 is also fixed to the holder 25. By this, the syringe 3 and the linear actuator 4 are fixed to the sled 24. The housing 13 can e.g. be fixed on a second sled 34 which can be fixed on holder 25 in different positions with regards to the distal direction x_{d} respectively the proximal direction xₚ of the syringe 3.

The linear actuator 4 comprises a cylindrical housing 35, a stamp 36, a driving unit 37 and a quadrature (rotary) encoder 38. The stamp 36 can slide into the cylindrical housing 35 in the proximal direction xₚ and out of the cylindrical housing 35 in the distal direction x_{d}. A distal end of the stamp 36 of the linear actuator 4 is fixed to a proximal end of the rod 14 of the syringe 3. The cylindrical housing 35 of the linear actuator 4 is fixed to the traverse 33 of the holder 25.

If the stamp 36 is moved in the distal direction x_{d}, the rod 14 of the syringe 3 is also moved in the distal direction x_{d}, and the volume of the chamber 16 within the housing 13 of the syringe 3 is decreased. As a result, fluid contained in the chamber 16 of the syringe 3 is pushed through the injection port 18 of the syringe 3 into the flexible tube 10 of the balloon catheter 2 where it displaces fluid into the balloon 11 and the balloon 11 of the balloon catheter 2 is inflated. By this, the linear actuator 5 is adapted for linearly moving the rod 14 of the syringe 3 in a distal direction x_{d} of the syringe 3 for injecting the fluid contained in the chamber 16 of the syringe 3 into the flexible tube 10, thereby increasing a volume of the fluid contained within the balloon 11.

If the stamp 36 is moved in the proximal direction xₚ, the rod 14 of the syringe 3 is also moved in the proximal direction xₚ, and the volume of the chamber 16 within the housing 13 of the syringe 3 is increased, thereby inducing a vacuum within the chamber 16. As a result, fluid contained in the flexible tube 10 of the balloon catheter 2 is sucked through the injection port 18 of the syringe 3 into the chamber 16 of the housing 13 of the syringe 3 and the flexible balloon 11 of the balloon catheter to is deflated. By this, the linear actuator 5 is adapted for linearly moving the rod 14 of the syringe 3 in a proximal direction xₚ of the syringe 3 for sucking in fluid contained in the flexible tube 10 into the chamber 16 of the syringe 3, thereby decreasing the volume of the fluid contained within the balloon 11.

The driving unit 37 in the form of a stepper motor drives the stamp 36 in small steps into the cylindrical housing 35 in the proximal direction xₚ and also in small steps out of the cylindrical housing 35 in the distal direction x_{d}. The stepper driver 45 of the first control unit 7 is communicatively connected to the pressure sensor 5 and the driving unit 37 of the linear actuator 4. A microprocessor P of the first control unit 7 receives the pressure data generated by the pressure sensor 5 and controls the stepper driver 45 which in turn controls the driving unit 37 of the linear actuator 4 depending on the received pressure data. In particular, the stepper driver 45 controls the number of steps by which the driving unit 37 drives the stamp 36 and the direction into which the stamp 36 is moved into the cylindrical housing 35 or out of the cylindrical housing 35.

As the balloon 11 and the flexible tube 10 of the balloon catheter 2 are retracted out of the aorta of the rabbit or the human being by use of the retracting unit 6 as described above, the pressure within the balloon 11 may vary, if a volume of the balloon 11 remains constant and the diameter of the aorta is changing. For example, an abdominal aorta can narrow from proximal to distal from about 5 mm to about 3 mm in diameter. At a constant balloon volume this can introduce significant pressure differences from about 1 bar to 3 bar when retracting the balloon 11 out of the aorta. As the balloon 11 is in contact with the aorta and is exerting a force on the aorta, wherein the force corresponds to the pressure within the balloon 11 respectively the balloon inflation pressure, a change of the inflation pressure of the balloon 11 results in a respective variation of the force exerted on the aorta by the balloon 11. When being inserted into the aorta of the rabbit, this leads to heterogeneous invasions and plaque development in the art of the rabbit. When being inserted into the aorta of the human being, this can lead to unintended lesions of the aorta of the human being.

To solve these problems, the microprocessor P of the first control unit 7 controls the stepper driver 45 which in turn controls the number of steps by which the driving unit 37 drives the stamp 36 and the direction into which the stamp 36 is moved into the cylindrical housing 35 or out of the cylindrical housing 35 in such a way, that the inflation pressure of the balloon 11 is kept stable at a pre-defined inflation pressure value. This is done by adapting the volume of the fluid contained within the balloon 11 to the changing diameter of the aorta. For example, an intended predefined inflation pressure value may be stored in the memory unit 20 of the first control unit 7. This pressure value can be in a range high enough to cause the balloon 11 to generate intended lesions in the aorta of the rabbit, if the balloon 11 has an inflation pressure as high as the predefined inflation pressure value and if the balloon catheter 2 is inserted into the aorta of the rabbit. The intended predefined pressure value alternatively can be in a range not high enough to cause the balloon 11 to generate lesions in the aorta of the human being, if the balloon 11 has an inflation pressure as high as the predefined inflation pressure value and if the balloon catheter 2 is inserted into the aorta of the human being.

The first control unit 7 may receive first pressure data representing a first inflation pressure of the balloon 11 and second pressure data representing a second inflation pressure of the balloon 11 from the pressure sensor 5. The second inflation pressure of the balloon 11 may be higher than the first inflation pressure of the balloon 11.

This pressure difference may be due to a decrease in the diameter of the aorta of the rabbit or the human being. The microprocessor P of the first control unit 7 controls the stepper driver 45 and is adapted for determining and controlling a direction as well as a number of steps by which the linear actuator 4 has to move the rod 14 of the syringe 3 in order to decrease the volume within the balloon 11, such that the second inflation pressure is decreasing towards the first inflation pressure. In this case, the microprocessor P of the first control unit 7 controls the stepper driver 45 and will determine and control the driving unit 37 in such a way that the stamp 36 of the linear actuator 4 is moved into the proximal direction, thereby forcing the rod 14 also to move in the proximal direction x_{d}. By this, the volume of the fluid contained within the balloon 11 can be decreased as well as the pressure of the balloon 11 as described above.

The second inflation pressure of the balloon 11 also may be lower than the first inflation pressure of the balloon 11. This pressure difference may be due to an increase in the diameter of the aorta of the rabbit or the human being. The microprocessor P of the first control unit 7 controls the stepper driver 45 and is adapted for determining and controlling a direction as well as a number of steps by which the linear actuator 4 has to move the rod 14 of the syringe 3 in order to increase the volume within the balloon 11, such that the second inflation pressure is increasing towards the first inflation pressure. In this case, the microprocessor P of the first control unit 7 controls the stepper driver 45 and will determine and control the driving unit 37 in such a way that the stamp 36 of the linear actuator 4 is moved into the distal direction x_{d}, thereby forcing the rod 14 also to move in the distal direction x_{d}. By this, the volume of the fluid contained within the balloon 11 can be increased as well as the pressure of the balloon 11 as described above.

A respective algorithm for controlling the driving unit 37 may be stored in the memory unit 20 of the first control unit 7. For example, the algorithm can be uploaded on and stored in the Arduino UNO board on which the first control unit 7 is mounted.

### Storing (Recording)/Plotting of Pressure Data and Step Data

The quadrature (rotary) encoder 38 is communicatively connected to the driving unit 37 of the linear actuator 4. In particular, the encoder 38 is adapted for receiving data from the driving unit 37 with regards to the number of steps the driving unit 37 has moved the stamp 36 and an according direction into which the stamp 36 has been moved by the driving unit 37. The encoder 38 is also communicatively connected to the encoder and recording unit 9. In particular, the encoder 38 can transmit data which it has received from the driving unit 37 to the encoder and recording unit 9 as described above. The encoder and recording unit 9 can encode and store the data received from the encoder 37 in the memory unit 22 of the encoder and recording unit 9. Additionally, the encoder and recording unit 9 can also be communicatively connected to the pressure sensor 5 (not shown). In particular, the encoder and recording unit 9 can receive and encode pressure data which have been generated by the pressure sensor 5 (e.g. the same pressure data which the pressure sensor 5 is transmitted to the first control unit 7).

If the encoder and recording unit 9 has received data from the encoder 38, wherein the data represents a number of steps and a respective direction into which the stamp 36 has been moved by the driving unit 37, the encoder and recording unit 9 can calculate a volume of the balloon 11 or a change of volume of the balloon 11. In other words, the number of steps and the direction can be calculated into a volume of the balloon 11 respectively a change of volume of the balloon 11. For example, the driving unit 37 can drive the stamp 36 with 0.0121 mm/step. An inner diameter of the chamber 13 of the syringe 3 can be for example 4.7 mm. If the driving unit 37 drives the stamp 36 for example 800 steps, the encoder and recording unit 9 can calculate the volume of the balloon 11 of the balloon catheter 2 as follows: 0.0121 mm/step*800 steps*(4.7 mm)² pi/4=168 μL. The flexible tube 10 can have for example a maximum capacity for fluid of 750 μL.

The encoder and recording unit 9 can also be communicatively connected to a computer unit 39 which comprises a fourth storing unit 40. In particular, the encoder and recording unit 9 can transmit the step data received by the encoder 38 and the pressure data received by the pressure sensor 5 to the computer unit 39. Similar to the encoder and recording unit 9 the computer unit 39 can store the pressure step data and pressure data in the fourth memory unit 40. Especially, the computer unit 39 can create real-time plots of the step data and the pressure data stored in the fourth memory unit 40. Aforementioned functions of the computer unit 39 can also be fulfilled by the single control unit 19 into which the computer unit 39 can be integrated, too.

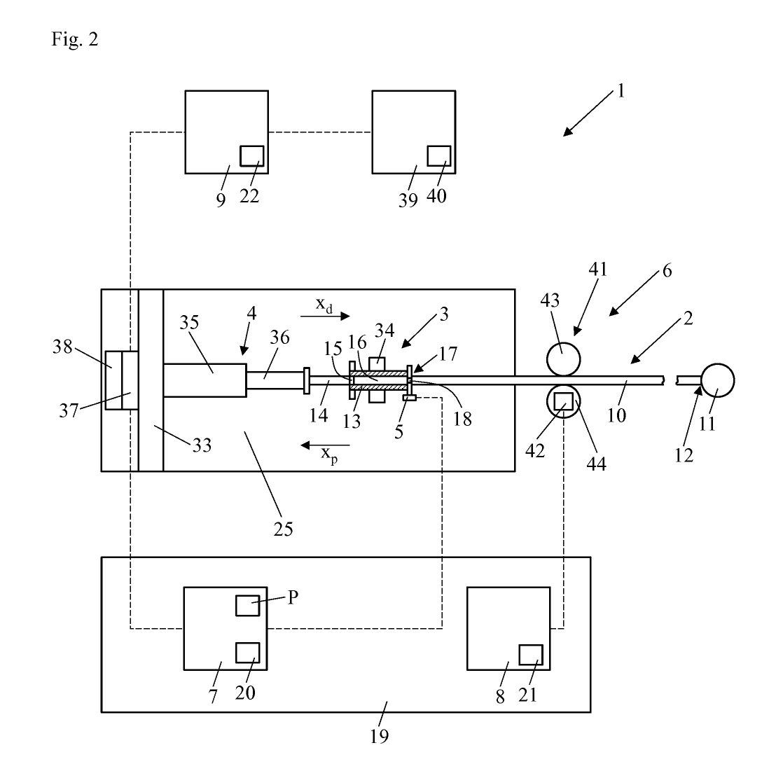

FIG. 2 shows a balloon computer device 1 according to another embodiment of the present invention. The device 1 is similar to that shown by FIG. 1. The difference mainly lies in the configuration of the balloon retracting unit 6. In the following, only differences between the devices 1 as per FIG. 1 and FIG. 2 are described in order to avoid repetitions.

The balloon retracting unit 6 as per FIG. 2 comprises a retracting roll assembly 41 and an electric DC gear motor 42. The retracting roll assembly 41 comprises a first roller wheel 43 and a second roller wheel 44 and advances the flexible tube 10 of the balloon catheter 2 such that the flexible tube 10 is retracted out of the aorta when driven by the motor 42. In detail, the flexible tube 10 is guided and quenched smoothly in-between the roller wheels 43 and 44, such that a rotational movement of the roller wheels 43 and 44—by friction between the wheels 43 and 44 and the flexible tube 10—leads to a movement of the flexible tube 10 in the distal direction x_{d} or in the proximal direction xₚ, depending on the turning direction of the roller wheels 43 and 44. For example, if the second roller wheel 44 is turning in a clockwise direction with regards to the view as per FIG. 2, the flexible tube 10 is moved by the roller wheels 43 and 44 in the distal direction x_{d}. If, on the other hand, the second roller wheel 44 is turning in an anti-clockwise direction with regards to the view as per FIG. 2, the flexible tube 10 is moved by the roller wheels 43 and 44 in the proximal direction xₚ. By this, the retracting roll assembly 41 can advance the flexible tube 10 of the balloon catheter 2.

The DC gear motor 42 is coupled to the second roller wheel 44, such that the motor 42 can drive the second roller wheel 44 into opposite turning directions (clockwise and anticlockwise according to the view as per FIG. 2) at a rotating speed. As the flexible tube 10 is moved in the proximal direction xₚ or in the distal direction x_{d}, the first roller wheel 43, which is in frictional contact with the flexible tube 10 is rotating in a turning direction, which is opposite to the turning direction of the second roller wheel 44. The DC gear motor 42 is communicatively connected to the second control unit 8. In particular, the second control unit 8 controls the motor 42, such that the motor 42 drives the second roller wheel 44 at a constant speed. As a result, the rotating speed of the retracting roll assembly is constant. By this, the flexible tube 10 especially can be advanced in the proximal direction xₚ such that the balloon 11 and the flexible tube 10 of the catheter 2 are retracted out of the aorta of the rabbit or the human being at a constant speed.

### Threshold Technique

FIG. 1 and FIG. 2 each show a device 1 comprising a first control unit 7 being capable to hold a stable predefined balloon inflation pressure over the whole retraction distance of the balloon catheter 2 out of the aorta of the rabbit or the human being. The balloon inflation pressure is measured by the pressure sensor 5. The linear actuator 4 is coupled to the syringe 3 and adjusts the volume of the balloon 11 (filling/emptying) depending on the actual balloon inflation pressure. The chamber 16, the flexible tube 10 and the balloon 11 are filled with the fluid, for example NaCl. The predefined balloon inflation pressure may be hold stable by a threshold technique, which is described in the following.

FIG. 3 shows a first diagram in the upper part of FIG. 3 and a second diagram in the lower part of FIG. 3. The first diagram comprises a horizontal axis (time in seconds) and a vertical axis (balloon inflation pressure in bar measured by the pressure sensor 5 and transmitted to the first control unit 7). The pressure sensor 5 continuously determines the pressure within the balloon 11 (balloon inflation pressure p), generates respective pressure data, and transmits the pressure data to the first control unit 7 as described above in conjunction with FIGS. 1 and 2. The chronological sequence of the balloon inflation pressure p is shown by the first diagram. The second diagram comprises a horizontal axis (time in seconds) and a vertical axis with 2 different values (value “1” representing a “state 1” in which the balloon 11 is inflated as described above in conjunction with FIGS. 1 and 2 by filling the balloon 11 with the fluid contained within the chamber 16 of the syringe and the flexible tube 10; value“0” representing a “state 0” in which the balloon 11 is deflated as described above in conjunction with FIGS. 1 and 2 by sucking out fluid contained within the balloon 11 into the flexible tube 10 and into the chamber 16 of the syringe 3). The chronological sequence of state 1 and state 0 is shown by the second diagram.

The horizontal axis of the first diagram and the second diagram run parallel to each other, wherein a certain time on the horizontal axis of the first diagram corresponds to the same time on the horizontal axis of the second diagram, meaning that for example a value of 2 seconds on the horizontal axis of the first diagram is situated vertical above a value of 2 seconds on the horizontal axis of the second diagram.

In the memory unit 20 of the first control unit 7 especially three values are stored: a predefined upper limit value of the balloon inflation pressure pᵤ (e.g. 1.2 bar), a predefined lower limit value of the balloon inflation pressure Pₗ (e.g. 1.0 bar) and a predefined actuating speed v [steps of the driving unit 37/per second] (e.g. 150 steps/sec) of the linear actuator 4.

The main principle of the threshold technique as depicted by FIG. 3 is that the microprocessor P of the first control unit 7 controls the stepper driver 45 and therefore the microprocessor P compares the pressure data which are generated by the pressure sensor 5 with the upper limit value of the balloon inflation pressure pᵤ and with the lower limit value of the balloon inflation pressure pₗ. Based on the result of such comparisons the stepper driver 45 controls the driving unit 37 of the linear actuator 4 in such a way that the balloon 11 is either inflated or deflated as described above in conjunction with FIGS. 1 and 2.

In a first comparison, the pressure data are compared with the lower limit value of the balloon inflation pressure pₗ. It is checked, whether a balloon inflation pressure value generated by the pressure sensor 5 exceeds the lower limit value of the balloon inflation pressure pₗ (requirement 1). If requirement 1 is fulfilled, the stepper driver 45 controls the driving unit 37 of the linear actuator 4 in such a way that the balloon 11 is deflated as described above in conjunction with FIGS. 1 and 2. This could be implemented in a computer program e.g. as follows: “If p (e.g. 1.1 bar)>pₗ (e.g. =1.0 bar), then “state 0”.

In a second comparison, the pressure data are compared with the upper limit value of the balloon inflation pressure pᵤ. It is checked, whether the balloon inflation pressure value received by the pressure sensor 5 is below the upper limit value of the balloon inflation pressure pᵤ (requirement 2). If requirement 2 is fulfilled, the stepper driver 45 controls the driving unit 37 of the linear actuator 4 in such a way that the balloon 11 is inflated as described above in conjunction with FIGS. 1 and 2. This could be implemented in a computer program e.g. as follows: “If p (e.g. 1.1 bar)<pᵤ (e.g. =1.2 bar), then “state 1”.

If requirement 1 is fulfilled but not requirement 2, the stepper driver 45 controls the driving unit 37 of the linear actuator 4 in such a way that the balloon 11 is deflated (state 0). If requirement 2 is fulfilled but not requirement 1, the stepper driver 45 controls the driving unit 37 of the linear actuator 4 in such a way that the balloon 11 is inflated (state 1). If both requirements 1 and 2 are fulfilled, the stepper driver 45 controls the driving unit 37 of the linear actuator 4 in such a way that the balloon 11 is inflated (state 1).

Referring now to FIG. 3, at the beginning (i.e. starting from 0 s) the balloon inflation pressure p is rising but still below the lower limit value of the balloon inflation pressure pₗ. Requirement 1 is not fulfilled but requirement 2. Therefore, the stepper driver 45 controls the driving unit 37 of the linear actuator 4 in such a way that the balloon 11 is inflated (state 1).

In its further course, at a first point in time t₁ the graph of the balloon inflation pressure p crosses a first auxiliary horizontal dashed line representing the lower limit value of the balloon inflation pressure pₗ. At this point of time t₁, the balloon inflation pressure p equals the lower limit value of the balloon inflation pressure pₗ. Requirement 1 is not fulfilled but requirement 2. Therefore, the stepper driver 45 controls the driving unit 37 of the linear actuator 4 in such a way that the balloon 11 is inflated (state 1). In its further course, up to but not including a second point in time t₂ the balloon inflation pressure exceeds the lower limit value of the balloon inflation pressure pₗ but still is below the upper limit value of the balloon inflation pressure pᵤ. Therefore, both requirements 1 and 2 are fulfilled and the balloon 11 is inflated.

However, at the second point in time t₂, the graph of the balloon inflation pressure p crosses a second auxiliary horizontal dashed line representing the upper limit value of the balloon inflation pressure pᵤ. At this point of time t₂, the balloon inflation pressure p equals the upper limit value of the balloon and inflation pressure pᵤ. Requirement 1 is fulfilled but not requirement 2. Therefore, the stepper driver 45 controls the driving unit 37 of the linear actuator 4 in such a way that the balloon 11 is deflated (state 0).

In its further course, the graph of the balloon inflation pressure p is still rising up to a third point in time t₃, although the balloon is already being deflated (state 0). This is due to the fact that a change from inflation to deflation influences the inflation pressure of the balloon 11 with some delay because of a delay duration in the system (it takes some time until a movement of the rod 14 of the syringe 3 in the proximal direction xₚ leads to a decrease in pressure within the balloon 11) respectively due to a loop time, i.e. a time period until the conditions are queried again.

At the third point of time t₃ the delay duration is over and the balloon inflation pressure p starts to decrease. At a fourth point of time t₄ the balloon inflation pressure p equals the upper limit value of the balloon inflation pressure pᵤ again. Therefore, at the fourth point of time t₄ the same conditions apply as at the second point of time t₂ (state 0).

As the balloon pressure value further decreases, both conditions 1 and 2 are fulfilled in-between the fourth point of time t₄ and a fifth point of time t₅. Therefore, the balloon 11 is inflated again (state 1). In-between the fourth point of time t₄ and a fifth point of time t₅ the graph of the balloon inflation pressure p is still declining up to the fifth point of time t₅, although the balloon is already being inflated again (state 1). This is due to the fact that a change from deflation to inflation influences the inflation pressure of the balloon 11 with some delay because of a delay duration in the system (it takes some time until a movement of the rod 14 of the syringe 3 in the distal direction x_{d} leads to an increase in pressure within the balloon 11) respectively due to a loop time, i.e. a time period until the conditions are queried again. At the fifth point of time t₅ the delay duration is over and the balloon inflation pressure p starts to increase again. At a sixth point of time t₆ the balloon inflation pressure p equals the upper limit value of the balloon inflation pressure pᵤ again. Therefore, at the sixth point of time t₆ the same conditions apply as at the fourth point of time t₄ and at the second point of time t₂ (state 0).

In its further course, the graph of the balloon inflation pressure p describes a zigzag course around the upper limit value of the balloon inflation pressure pᵤ. A maximum deviation of the balloon inflation pressure p to the upper limit value of the balloon inflation pressure pᵤ in said zigzag course is a threshold value. The zigzag course is generated by the controlling of the driving unit 37 by the stepper driver 45 of the first control unit using the threshold technique as described above.

1. A balloon catheter device (1) for use in an aorta of a rabbit or a human being, comprising:
a balloon catheter (2),
a syringe (3),
an actuator (4),
a pressure sensor (5),
a balloon retracting unit (6),
a first control unit (7) and
a second control unit (8),
wherein the balloon catheter (2) comprises a flexible tube (10) and a balloon (11) at a distal end (12) of the tube (10),
wherein the syringe (3) comprises a chamber (16) containing a fluid, wherein the chamber (16) is connected to a proximal end of the tube (10),
wherein the actuator (4) is adapted
for injecting the fluid contained in the chamber (16) of the syringe (3) into the flexible tube (10), thereby increasing a volume of the fluid contained within the balloon (11), and
for sucking in fluid contained in the flexible tube (10) into the chamber (16) of the syringe (3), thereby decreasing the volume of the fluid contained within the balloon (11),
wherein the pressure sensor (5) is adapted for sensing an inflation pressure of the balloon (11),
wherein the balloon retracting unit (6) is adapted for retracting the balloon (11) out of the aorta,
wherein the first control unit (7) is communicatively connected to the pressure sensor (5) and the actuator,
wherein the second control unit (8) is communicatively connected to the balloon retracting unit (6),
wherein the first control unit (7) is adapted to receive pressure data representing the inflation pressure of the balloon (11) measured by the pressure sensor (5) and to control the actuator (4) depending on the received pressure data, such that the inflation pressure of the balloon (11) is kept stable at a predefined inflation pressure value, and
wherein the second control unit (8) is adapted to control the balloon retracting unit (6), such that the balloon (11) is retracted out of the aorta at a constant speed.

2. The balloon catheter device (1) according to claim 1,
wherein the balloon catheter (2) is a balloon catheter for generating reproducible minimal-invasive lesions in the aorta of the rabbit, and
wherein the predefined inflation pressure value is high enough to cause the balloon (11) to generate intended lesions in the aorta of the rabbit, if the balloon (11) has an inflation pressure as high as the predefined inflation pressure value.

3. The balloon catheter device (1) according to claim 1,
wherein the balloon catheter (2) is a balloon catheter for use in pressure controlled balloon thrombectomy on the human being, and
wherein the predefined inflation pressure value is not high enough to cause the balloon (11) to generate lesions in the aorta of the human being, if the balloon (11) has an inflation pressure as high as the predefined inflation pressure value.

4. The balloon catheter device (1) according to claim 1, wherein the pressure sensor (5) is a Luer lock pressure sensor.

5. The balloon catheter device (1) according to claim 1, further comprising:
an encoder and recording unit (9) and
a quadrature encoder (38),
wherein the encoder and recording unit (9) is adapted for receiving, encoding and storing pressure data representing measured pressures from the pressure sensor (5) and step data representing a number of steps performed by the actuator (4) from the quadrature encoder (38).

6. The balloon catheter device (1) according to claim 5,
wherein the encoder and recording unit (9) is adapted for calculating a volume of the balloon (11) depending on the received pressure data and step data.

7. The balloon catheter device (1) according to claim 5, further comprising a computer unit (39),
wherein the encoder and recording unit (9) is adapted for transmitting the received pressure data and step data to the computer unit (39),
wherein the computer unit (39) is adapted for storing the transmitted pressure data and step data and for creating real-time plots of both the transmitted pressure data and step data.

8. The balloon catheter device (1) according to claim 1, wherein the first control unit (7) and the second control unit (8) are integrated into a single control unit (19).

9. The balloon catheter device (1) according to claim 8, wherein the encoder and recording unit (9) is integrated into the single control unit (19).

10. The balloon catheter device (1) according to claim 1,
wherein the balloon retracting unit (6) comprises
a rack (23),
a sled (24), and
driving means (26),
wherein the sled (24) is slideably mounted on the rack (23),
wherein the syringe (3) and the linear actuator (4) are fixedly mounted on the sled (24),
wherein the driving means (26) are adapted for driving the sled (24) such that the sled (20) is sliding along the rack (23) at a sliding speed, and
wherein the second control unit (8) can control the driving means (26), such that the sliding speed of the sled (24) is constant.

11. The balloon catheter device (1) according to claim 10,
wherein the rack (23) comprises two guiding rods (27, 28),
wherein the driving means (26) comprise a rotating stepper (29) and a spindle drive (30),
wherein the sled (24) is guided in a linear direction (xₚ, x_{d}) by means of the two guiding rods (27, 28),
wherein the spindle drive (30) can drive the sled (24) such that the sled (24) is sliding along the two guiding rods (27, 28) at the sliding speed, and
wherein the second control unit (8) can control the spindle drive (30), such that the sliding speed of the sled (24) is constant.

12. The balloon catheter device (1) according to claim 1,
wherein the balloon retracting unit (6) comprises
a retracting roll assembly (41) and
an electric motor (42), in particular a DC gear motor,
wherein the electric motor (42) is adapted for driving the retraction roll assembly (41) such that the retracting roll assembly (41) is rotating at a rotating speed,
wherein the retracting roll assembly (41) is configured for advancing the flexible tube (10) of the balloon catheter (2) such that the flexible tube (10) is retracted out of the aorta when driven by the electric motor (42), and
wherein the second control unit (8) is configured for controlling the electric motor (42), such that the rotating speed of the retracting roll assembly (41) is constant.

13. The balloon catheter device (1) according to claim 1,
wherein the first control unit (7) comprises a memory unit (20),
wherein a predefined upper limit value of the balloon inflation pressure is stored in the memory unit (20),
wherein a predefined lower limit value of the balloon inflation pressure is stored in the memory unit (20),
wherein a predefined actuating speed of the linear actuator (4) is stored in the memory unit (20),
wherein the first control unit (7) is adapted for deflating the balloon (11) by driving the linear actuator (4) at the predefined actuating speed, such that a rod (14) of the syringe (3) is moved in a proximal direction (xₚ) of the syringe (3) at a speed proportional to the predefined actuating speed and sucks in fluid contained in the flexible tube (10) into the chamber (16) of the syringe (3), thereby decreasing the volume of the fluid contained within the balloon (11), if a value of a measured inflation pressure is higher than the predefined lower limit value of the balloon inflation pressure, and
for inflating the balloon (11) by driving the linear actuator (4) at the predefined actuating speed, such that the rod (14) of the syringe (3) is moved in a distal direction (x_{d}) of the syringe (3) at a speed proportional to the predefined actuating speed and injects the fluid contained in the chamber (16) of the syringe (3) into the flexible tube (10), thereby increasing a volume of the fluid contained within the balloon (11), if the value of the measured inflation pressure is lower than the predefined upper limit value of the balloon inflation pressure,
wherein the first control unit (7) is adapted for inflating the balloon (11),
if the value of the measured inflation pressure is higher than the predefined lower limit value of the balloon inflation pressure and
if the value of the measured inflation pressure is lower than the predefined upper limit value of the balloon inflation pressure.

14. The balloon catheter device (1) according to claim 1,
wherein the pressure sensor (5) is adapted to sense the inflation pressure of the balloon (11) in real-time, and
wherein the balloon catheter device (1) is adapted to transmit the inflation pressure of the balloon (11) wirelessly to an external computer unit.
